(19) United States
(12) Patent Application Publication
(10) Pub. No.: US 2024/0315984 A1
(43) Pub. Date: Sep. 26, 2024

(54) MICROSPHERE-BASED DRUG DELIVERY PLATFORM FOR DELIVERY OF IMMUNOTHERAPEUTIC AGENTS

(71) Applicant: Boston Scientific Scimed, Inc., Maple Grove, MN (US)

(72) Inventors: Bhanu Prasanth Koppolu, Cary, NC (US); Philip David Dorgan, Cork (IE); Rhiannon Johnson, Maple Grove, MN (US); Steven L. Kangas, Woodbury, MN (US); Nivedita Swetha Ramkumar, Cork (IE); Matthew R. Dreher, Dover, MA (US)

(73) Assignee: Boston Scientific Scimed, Inc., Maple Grove, MN (US)

(21) Appl. No.: 18/612,755

(22) Filed: Mar. 21, 2024

Related U.S. Application Data
(60) Provisional application No. 63/492,063, filed on Mar. 24, 2023.

(51) Int. Cl. A61K 9/51; A61K 45/06

(52) U.S. Cl. CPC A61K 9/5153; A61K 9/5123; A61K 45/06

(57) ABSTRACT

The present disclosure pertains to biodegradable polymeric microspheres that comprise a biodegradable polymer and an immunotherapeutic agent selected from an inhibitor of poly ADP ribose polymerase enzyme (PARP inhibitor) and/or a Toll-like receptor (TLR) agonist wherein (a) 50% of a total amount of the immunotherapeutic agent in the biodegradable polymeric microspheres is released from the biodegradable polymeric microspheres into a 37° C. solution of PBS Tween 20 (0.05%) at a point in time ranging from 3 days and 14 days; (b) between 1 mg and 100 mg of immunotherapeutic agent per 1 g dry weight of microspheres is released from the biodegradable polymeric microspheres into a 37° C. solution of PBS Tween 20 (0.05%) at a point in time ranging from 3 days and 14 days; or (c) both (a) and (b). Other aspects of the present disclosure pertain to methods of using such microparticles and kits that contain such microparticles.

### CROSS-REFERENCE TO RELATED APPLICATIONS

This application claims the benefit of U.S. Provisional Patent Application Ser. No. 63/492,063 filed on Mar. 24, 2023, the disclosure of which is incorporated herein by reference.

### BACKGROUND

Immunotherapies have fast become the standard of care in many cancer indications. However, only a minority of the patients respond to the therapy. Recently, focus has shifted to development of more potent combination therapies and immunomodulators. Many of these next-generation immunotherapies are known to induce robust antitumor immune responses. However, systemic delivery of these immunotherapeutic agents is hindered by strong off-target toxicity. As such, there is a need for a drug delivery system that can deliver an immunotherapeutic agent to a target tumor in a targeted and controlled manner, thereby reducing or eliminating off-target toxicity while improving efficacy.

### SUMMARY

In some aspects, the present disclosure pertains to microspheres comprising a biodegradable polymer and an immunotherapeutic agent selected from an inhibitor of poly ADP ribose polymerase enzyme (PARP inhibitor) and/or a Toll-like receptor (TLR) agonist, wherein 50% of a total amount of the immunotherapeutic agent in the biodegradable polymeric microspheres is released at 37° C. from the biodegradable polymeric microspheres into a PBS Tween 20 (0.05%) solution at a point in time ranging from 3 days and 14 days

In some aspects, which can be used in conjunction with the above aspects, the present disclosure pertains to microspheres comprising a biodegradable polymer and an immunotherapeutic agent selected from an inhibitor of poly ADP ribose polymerase enzyme (PARP inhibitor) and/or a Toll-like receptor (TLR) agonist, wherein from 1 mg/g dry weight of microspheres to 100 mg/g dry weight of microspheres is released at 37° C. from the biodegradable polymeric microspheres into a PBS Tween 20 (0.05%) solution at a point in time ranging from 3 days and 14 days.

In some embodiments, which can be used in conjunction with the above aspects, the immunotherapeutic agent is dispersed throughout the particles in the form of nanoparticles having a size ranging from 100 nm to 2000 nm.

In some embodiments, which can be used in conjunction with the above aspects and embodiments, the biodegradable polymeric microspheres contain between 3 wt % and 10 wt % of the immunotherapeutic agent based on a dry weight of the biodegradable polymeric microspheres.

In some embodiments, which can be used in conjunction with the above aspects and embodiments, the biodegradable polymeric microspheres range from 30 to 200 microns in diameter.

In some embodiments, which can be used in conjunction with the above aspects and embodiments, the immunotherapeutic agent is released by a bulk erosion and hydrolysis.

In some embodiments, which can be used in conjunction with the above aspects and embodiments, the biodegradable polymer is a biodegradable polyester.

In some embodiments, which can be used in conjunction with the above aspects and embodiments, the biodegradable polymer consists of or comprises poly(lactide-co-glycolide) (PLGA). In some of these embodiments, a ratio of lactide to glycolide units in the PLGA is between 60:40 and 40:60, typically, 50:50. In some of these embodiments, the PLGA comprises acid terminated PLGA or the PLGA comprises a combination of acid terminated PLGA and ester terminated PLGA.

In some embodiments, which can be used in conjunction with the above aspects and embodiments, the biodegradable polymeric microspheres further comprise a pharmaceutical excipient selected from the group consisting of a preservative, a tonicity adjusting agent, a viscosity adjusting agent, a pH adjusting agent, a contrast agent, a detergent, and/or a sugar or sugar alcohol.

In some embodiments, which can be used in conjunction with the above aspects and embodiments, the biodegradable polymeric microspheres are provided in dry form.

In some embodiments, which can be used in conjunction with the above aspects and embodiments, the biodegradable polymeric microspheres are provided in an aqueous liquid.

In some embodiments, which can be used in conjunction with the above aspects and embodiments, the biodegradable polymeric microspheres are provided in a vial or a syringe.

In some aspects, the present disclosure pertains to kits that comprise (a) biodegradable polymeric microspheres in accordance with the above aspects and embodiments; and (b) any one, any two, any three, any four, any five or all six of the following items: a syringe barrel, a vial, a needle, catheter, a guide wire, or an injectable liquid.

In some aspects, the present disclosure pertains to methods for the treatment of a patient having a solid tumor, comprising delivering to the solid tumor biodegradable polymeric microspheres in accordance with the above aspects and embodiments.

In some embodiments, the biodegradable polymeric microspheres are delivered by direct injection into the tumor.

In some embodiments, the biodegradable polymeric microspheres are delivered via one or more blood vessels feeding at least part of the tumor and at least a portion of the biodegradable polymeric microspheres lodge in the blood vessels to provide an embolus.

In some embodiments, which can be used in conjunction with the above aspects and embodiments, the method further comprises treating the solid tumor with radiation therapy.

These and other aspects, embodiments and advantages of the present disclosure will become immediately apparent to those of ordinary skill in the art upon review of the detailed description and claims to follow.

### BRIEF DESCRIPTION OF THE FIGURES

FIG. 1 is a micrograph of biodegradable polymeric microspheres produced in accordance with an embodiment of the present disclosure.

FIG. 2 is a graph showing kinetic drug release into PBS Tween at 37° C. from imiquimod-containing biodegradable polymeric microspheres and olaparib-containing biodegradable polymeric microspheres, in accordance with embodiments of the present disclosure.

### DETAILED DESCRIPTION

In various aspects, the present disclosure pertains to biodegradable polymeric microspheres that are loaded with an immunotherapeutic agent of interest. The biodegradable polymeric microspheres are useful for the delivery of immunotherapeutic agents to solid tumors, for example, by direct injection or as embolic agents that are delivered via one or more feeder vessels to the tumor. This approach allows for controlled targeted delivery of potent immunotherapeutic agents, thereby reducing off-site toxicity while improving target site efficacy.

In some embodiments, the present disclosure pertains to biodegradable polymeric microspheres that comprise a biodegradable polymer and an immunotherapeutic agent wherein 50% of the total amount of the immunotherapeutic agent in the biodegradable polymeric microspheres is released from the biodegradable polymeric microspheres into a 37° C. solution of PBS Tween 20 (0.05%) at a point in time ranging from 3 days and 14 days. In some embodiments, less than 50%, less than 40%, less than 30%, or less than 25% of the immunotherapeutic agent is released at 3 days and/or greater than 50%, greater than 60%, greater than 70%, or greater than 80% of the immunotherapeutic agent is released at 14 days.

In some embodiments, the present disclosure pertains to biodegradable polymeric microspheres that comprise a biodegradable polymer and an immunotherapeutic agent wherein between 1 mg and 100 mg of immunotherapeutic agent per 1 g dry weight of microspheres (e.g., ranging anywhere from 1 mg to 2 mg to 5 mg to 10 mg to 25 mg to 50 mg to 100 mg of immunotherapeutic agent per 1 g dry weight of microspheres) is released from the biodegradable polymeric microspheres into a 37° C. solution of PBS Tween 20 (0.05%) at a point in time ranging from 3 days and 14 days.

In some embodiments, immunotherapeutic agents for use in the present disclosure include poly(ADP-ribose) polymerase (PARP) inhibitors and/or Toll-like receptor (TLR) agonists.

Examples of PARP inhibitors include olaparib (AZD-2281), rucaparib (PF-01367338), niraparib (MK-4827), talazoparib (BMN-673), veliparib (ABT-888), CEP 9722, E7016, BGB-290, and 3-aminobenzamide, among others.

Examples of TLR agonists include TLR3, TLR4, TLR7, TLR8 and TLR9 agonists. More particular TLR agonists include TLR 7/8 agonists selected from imidazoquinoline derivatives (IMDs) (e.g., imiquimod, resiquimod (R848), gardiquimod, 852-A (PF-4878691), MEDI9197, etc.), pteridinone-based derivatives (e.g., vesatolimod GS-9620), 8-oxoadenine derivatives (e.g., AZD-8848, etc.), TMX-202, benzazepine analogs (e.g., motolimod (VTX-2337), etc.), and pyrimidine analogs (e.g., selgantolimod GS-9688, etc.), among others. More particular TLR 9 agonists include CpG oligodeoxynucleotides (e.g., agatolimod, etc.), lefitolimod (MGN1703), and tilsotolimod, among others.

In some embodiments, immunotherapeutic agents for use in the present disclosure include small molecule (i.e., having a molecular weight that is less than 3000 Daltons) PARP inhibitors and/or TLR agonists.

In some embodiments, the immunotherapeutic agent is dispersed throughout the biodegradable polymeric microspheres in particulate form. For example, the immunotherapeutic agent may be in the form of nanoparticles having a size ranging from 100 nm to 2000 nm, more typically 250 nm to 1000 nm, among other sizes. Such particle sizes may be achieved, for example, by milling the immunotherapeutic agent to a desired size.

Biodegradable polymeric microspheres can be made by a number of techniques known to those skilled in the art, such as single and double emulsion techniques, solvent evaporation techniques, suspension polymerization techniques, solvent extraction techniques, and combinations thereof, among others. In particular embodiments including those described herein, biodegradable polymeric microspheres are made by emulsion-solvent evaporation techniques. The biodegradable polymeric microspheres of the present disclosure are particularly useful for embolization, as sizes of microspheres can be controlled, for example by sieving or other classifying/fractionating techniques. Moreover, unwanted aggregation may be avoided due to the spherical shape of the biodegradable polymeric microspheres.

The biodegradable polymeric microspheres may have a range of loading levels. In some embodiments, the biodegradable polymeric microspheres contain between 1 and 20 wt % (dry weight) of the immunotherapeutic agent, more typically, between 3 and 10 wt % (dry weight) of the immunotherapeutic agent.

The biodegradable polymeric microspheres may have a wide range of particle sizes. For example, the biodegradable polymeric microspheres may range from 10 microns to 500 microns in diameter, typically ranging from 30 to 200 microns in diameter, more typically ranging from 60 to 150 microns in diameter.

The biodegradable polymeric microspheres may biodegrade in vivo by a variety of processes including bulk erosion and surface erosion.

The biodegradable polymeric microspheres comprise one or more types of biodegradable polymers. In some embodiments, the biodegradable polymers degrade by hydrolysis. In some embodiments, the biodegradable polymers are biodegradable polyesters.

Examples of biodegradable polyesters include polylactides such as polylactide (PLA), polyglycolides such as polyglycolide (PLG), polyhydroxyalkanoates such as polyhydroxybutyrate or polyhydroxyvalerate, polylactones such as poly-γ-butyrolactone, poly-δ-valerolactone or poly-ε-caprolactone, poly(alkenedicarboxylates) such as poly(butylene succinate) or poly(butylene adipate), poly(p-dioxanone), poly(trimethylene carbonate), as well as copolymers containing two, three or more of any combination of monomers in the preceding polymers, including poly(lactide-co-glycolide) (PLGA), poly(3-hydroxybutyrate-co-3-hydroxyvalerate), and poly(butylene succinate-co-adipate), among others.

In some embodiments, the biodegradable polymeric microspheres contain between 80 and 99 wt % (dry weight) of one or more biodegradable polyesters, more typically, between 90 and 97 wt % (dry weight) of one or more biodegradable polyesters.

Biodegradable polyesters may be employed in a variety of molecular weights, for example having a number average molecular weight ranging, for example, from 500 to 250,000 Daltons, in some embodiments, from 1000 to 20,000 Daltons.

Biodegradable polyesters may be terminated with a variety of functional groups including polyesters that contain one or more carboxylic acid end groups (e.g., acid terminated PLGA, which contains a carboxylic acid end group), polyesters that contain one or more ester end groups (e.g., ester-terminated PLGA's including methyl-ester-terminated PLGA, ethyl-ester-terminated PLGA, propyl-ester-terminated PLGA, etc.), or one or more hydroxyl groups.

Biodegradable copolyesters may have a variety of monomer ratios. For example, biodegradable copolyesters having two monomers may have monomer ratios that range, for example, from 1:99 to 10:90 to 25:75 to 35:65 to 50:50 to 65:35 to 75:25 to 90:10 to 99:1 (i.e., ranging between any two of the preceding ratios).

In certain beneficial embodiments, the biodegradable polymeric microspheres comprise two more types of biodegradable polyesters. Examples include (a) combinations of biodegradable polyesters that are formed from different monomers (e.g., a combination of PLA and PLGA, etc.), (b) combinations of biodegradable polyesters that are formed from the same monomer(s) but have different molecular weights, (c) combinations of biodegradable polyesters that are formed from the same monomer(s) but have different terminal groups (e.g., a combination of carboxylic acid terminated PLGA and ester-terminated PLGA, etc.), (d) combinations of biodegradable co-polyesters that are formed from the same monomers but have different ratios of the monomers (e.g., a combination of 50:50 PLGA and 75:25 PLGA, etc.), (e) a combination of any two or all three of the preceding combinations (b), (c) and (d).

The biodegradable polymeric microspheres of the present disclosure may be stored and transported as a sterile dry composition. The dry composition may be shipped, for example, in a syringe, vial, ampoule, catheter, or other container (e.g., any container that is configured to interact with a needle or delivery catheter). The dry composition may be mixed with a suitable liquid carrier (e.g. sterile water for injection, physiological saline, phosphate buffer, a solution containing an imaging contrast agent, etc.) prior to administration. In this way the concentration of the biodegradable polymeric microspheres in the composition to be injected may be varied at will, depending on the specific application at hand, as desired by the health care practitioner in charge of the procedure. One or more containers of liquid carrier may also be supplied and shipped, along with the dry particles, in the form of a kit.

As long as unacceptable levels of degradation do not occur (which is related to polymer properties, solution properties, shelf-life requirements, etc.), the biodegradable polymeric microspheres may be stored in a sterile liquid suspension that includes the biodegradable polymeric microspheres and an aqueous or non-aqueous liquid. As with the dry composition, the liquid suspension may be stored, for example, in a syringe, vial, ampoule, catheter, or other container. The liquid suspension may also be mixed with a suitable liquid carrier (e.g. sterile water for injection, physiological saline, phosphate buffer, a solution containing contrast agent, etc.) prior to administration, allowing the concentration of biodegradable polymeric microspheres in the suspension to be administered to be reduced prior to injection, if so desired by the health care practitioner in charge of the procedure. One or more containers of liquid carrier may also be supplied to form a kit.

Dry compositions or liquid suspensions that contain the biodegradable polymeric microspheres may also optionally contain additional agents, for example, selected from imaging agents, tonicity adjusting agents, suspension agents, wetting agents, pH adjusting agents and colorants. Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxyl or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents such as metallic particles, for example, particles of tantalum, tungsten, rhenium, niobium, molybdenum, and their alloys, which metallic particles may be spherical or non-spherical, non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of additional agents further include tonicity adjusting agents such as sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others, suspension agents including various surfactants, wetting agents, and polymers (e.g., albumen, PEO, polyvinyl alcohol, block copolymers, etc.), among others, and pH adjusting agents including various buffer solutes. Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

In various embodiments, kits are provided that include one or more delivery devices for delivering the biodegradable polymeric microspheres of the present disclosure to a subject. Such kits may include one or more of the following: a syringe barrel, which may or may not contain biodegradable polymeric microspheres as described hereinabove; a vial, which may or may not contain biodegradable polymeric microspheres as described hereinabove; a needle; a flexible tube (e.g., a catheter); a guide wire; and an injectable liquid such as water for injection, normal saline, phosphate buffered saline, a solution containing an imaging contrast agent, etc. Where supplied, the catheter may be used to inject the biodegradable polymeric microspheres into a blood vessel of a patient, and the guide wire may be used to position the catheter within the blood vessel. Whether supplied in a syringe, vial, or other reservoir, the biodegradable polymeric microspheres may be provided in a form of a dry composition or a liquid suspension as described above.

In various embodiments, compositions comprising the biodegradable polymeric microspheres of the present disclosure are sterile. The compositions can be sterilized by any method known in the art, for example, by irradiation, such as gamma or beta or Xray irradiation. In certain embodiments, the biodegradable polymeric microspheres are prepared aseptically using aseptic techniques.

In other aspects, the present disclosure provides methods for the treatment of patients having a solid tumor by delivering compositions comprising the biodegradable polymeric microspheres of the present disclosure (e.g., a liquid suspension of the biodegradable polymeric microspheres) to the solid tumor. Examples of solid tumors include solid tumors such as liver tumors, pancreatic tumors, uterine tumors, brain tumors, renal tumors, bone tumors, breast tumors, bladder tumors, prostate tumors, colon cancer tumors, lung tumors, mouth and/or neck tumors, esophageal tumors, endometrial tumors, ovarian tumors, gastric tumors and stomach tumors, among others. The amount of immunotherapeutic agent delivered to the solid tumor will vary, for example, depending upon the particular immunotherapeutic agent being administered, the type of tumor, the size of the tumor, the state of the patient, and the like. In some embodiments, the solid tumor may be further treated with radiation therapy.

Methods of administration of the biodegradable polymeric microspheres include, for example, percutaneous techniques as well as other effective routes of administration.

For example, in some embodiments, compositions comprising the biodegradable polymeric microspheres of the present disclosure may be delivered by direct injection into the tumor. For example, the biodegradable polymeric microspheres may be delivered by needle that is of size 24 gauge or below.

In some embodiments, compositions comprising the biodegradable polymeric microspheres of the present disclosure are delivered via one or more blood vessels feeding at least part of the tumor (e.g., a feeder artery), wherein at least a portion of the biodegradable polymeric microspheres lodge in the blood vessels to provide an embolus. For example, the biodegradable polymeric microspheres may be delivered, for example, through a catheter ranging in size from 1.2 French and above.

Once delivered to a tumor, immunotherapeutic agent is then released locally through hydrolysis-based biodegradation of the biodegradable polymeric microspheres. The immunotherapeutic agent is then delivered locally and in a controlled manner over a timeframe of days to weeks. This allows for the immunotherapeutic agent dose to be maintained within an immunotherapeutic window locally while minimizing systemic distribution.

### Example

The following PLGA products, available from Evonik Industries AG, Essen, Germany, are used in the present Example: RESOMER® RG 501 H, an acid terminated poly(D,L-lactide-co-glycolide) 50:50 molar ratio, having an inherent viscosity ranging from 0.8-0.16 dl/g; RESOMER® RG 502, an ester terminated poly(D,L-lactide-co-glycolide) 50:50 molar ratio, M_{w} 7,000-17,000, having an inherent viscosity ranging from 0.16-0.24 dl/g; RESOMER® RG 502 H an acid terminated poly(D,L-lactide-co-glycolide) 50:50 molar ratio, M_{w} 7,000-17,000, having an inherent viscosity ranging from 0.16-0.24 dl/g; and RESOMER® RG 503 H an acid terminated poly(D,L-lactide-co-glycolide) 50:50 molar ratio, having an inherent viscosity ranging from 0.16-0.24 dl/g.

2 ml of PLGA solution are formed by dissolving the PLGA (50:50 weight ratio RG 501 H:RG 502 for imiquimod microspheres; 50:50 weight ratio RG 501 H:RG 502 H (2A) or RG501 H:RG 503H (4A) for olaparib microspheres) in dichloromethane (DCM) in an amount of <25% w/v. The PLGA was dissolved at ambient temperature while stirring for 30 minutes.

Immunotherapeutic agent (imiquimod or olaparib) was milled to a size of 500-1000 nm in dichloromethane (DCM and added to the PLGA solution in varying amounts up to 12 w % of the PLGA in solution, forming an PLGA/immunotherapeutic organic solution.

5 ml of PVA solution are formed by dissolving a surfactant/stabilizing agent such as polyvinyl alcohol (PVA) in water (e.g., sterile or deionized water) in an amount of 2 wt %. The PVA was dissolved at up to 80° C. while vigorously stirring for one hour or until fully dissolved, forming a PVA aqueous solution.

The PLGA/immunotherapeutic organic solution is then added dropwise or through a gauged needle to the PVA aqueous solution at ambient temperature while stirring at 300 rpm. The resulting mixture is then subjected to homogenization for 5 minutes, thereby forming an oil-in-water emulsion. The emulsion is the diluted ^{˜}2× with sterile water and incubated at ambient temperature (25° C.) overnight while stirring at 150 rpm.

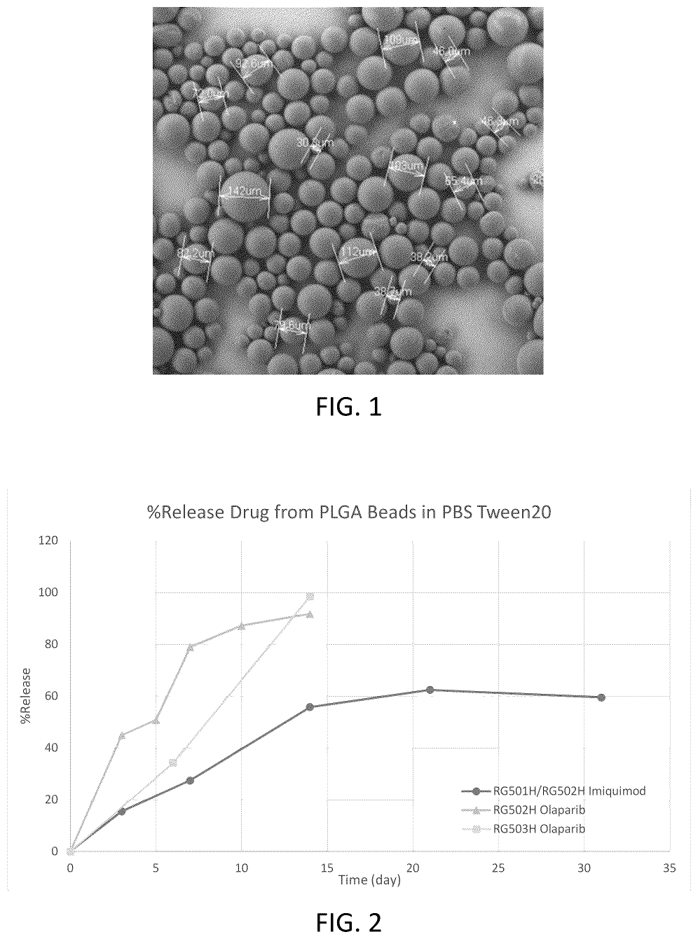

The resulting microsphere mixture is then added to 250 ml 1% PVA solution and incubated at 31° C. for 48 hours at 80 rpm. The liquid phase was decanted and the biodegradable polymeric microspheres were washed in 100 ml sterile water. This washing step was repeated 3 times. The resulting microspheres are shown in FIG. 1.

Microspheres were then filtered through a 150 micron sieve followed filtration by a 80 or 100 um woven nylon mesh, resulting in particles ranging from 80-150 microns or 100-150 microns. The classified particles were then dried under vacuum at ambient temperature for >12 hours.

Acetate buffer (pH ^{˜}4) was prepared by dissolving 0.93 g sodium acetate and 2.321 mL glacial acetic acid in 500 ml water.

Immunotherapeutic agent content of the biodegradable polymeric microspheres is measured by weighing ^{˜}1 mg of the biodegradable polymeric microspheres in a vial, adding 10 ml of 1:1 acetonitrile (ACN):acetate buffer, dissolving the biodegradable polymeric microspheres, running a UV-Visible spectroscopic analysis (for imiquimod, wavelength=241 nm or 319 nm; for olaparib, wavelength=253 nm) and using a slope of previously run immunotherapeutic agent standards to calculate the concentration of immunotherapeutic agent (ug/ml of solution). From that concentration, the % immunotherapeutic agent content of the biodegradable polymeric microspheres is calculated. Imiquimod content of the biodegradable polymeric microspheres was found to be ^{˜}7 wt %, and olaparib content of the biodegradable polymeric microspheres was found to be ^{˜}5 wt %.

Kinetic drug release from the biodegradable polymeric microspheres is measured by weighing out ^{˜} 1 mg of the biodegradable polymeric microspheres in a centrifuge tube, adding 10 mL PBS Tween and incubating on a shaker at 120 rpm at 37° C. For each time point of interest (e.g., 3, 7, 10, 14, 21 days), the biodegradable polymeric microspheres are centrifuged down and media pipetted off. Then, 5 ml of 1:1 ACN:Acetate buffer (pH 4) is added directly to the biodegradable polymeric microspheres and a UV-Visible spectroscopic analysis is performed. The measured immunotherapeutic concentration is used to calculate the % immunotherapeutic agent content remaining in the biodegradable polymeric microspheres. Kinetic drug release results are shown in FIG. 2.

1. Biodegradable polymeric microspheres comprising a biodegradable polymer and an immunotherapeutic agent selected from an inhibitor of poly ADP ribose polymerase enzyme (PARP inhibitor) and/or a Toll-like receptor (TLR) agonist wherein (a) 50% of a total amount of the immunotherapeutic agent in the biodegradable polymeric microspheres is released from the biodegradable polymeric microspheres into a 37° C. solution of PBS Tween 20 (0.05%) at a point in time between 3 days and 14 days; (b) between 1 mg and 100 mg of immunotherapeutic agent per 1 g dry weight of microspheres is released from the biodegradable polymeric microspheres into a 37° C. solution of PBS Tween 20 (0.05%) at a point in time between 3 days and 14 days; or (c) both (a) and (b).

2. The biodegradable polymeric microspheres of claim 1, wherein the immunotherapeutic agent is dispersed throughout the particles in the form of nanoparticles having a size ranging from 100 nm to 2000 nm.

3. The biodegradable polymeric microspheres of claim 1, wherein the biodegradable polymeric microspheres contain between 3 wt % and 10 wt % of the immunotherapeutic agent based on a dry weight of the biodegradable polymeric microspheres.

4. The biodegradable polymeric microspheres of claim 1, wherein the biodegradable polymeric microspheres range from 30 to 200 microns in diameter.

5. The biodegradable polymeric microspheres of claim 1, wherein the immunotherapeutic agent is released by a bulk erosion and hydrolysis.

6. The biodegradable polymeric microspheres of claim 1, wherein the biodegradable polymer is a biodegradable polyester.

7. The biodegradable polymeric microspheres of claim 1, wherein the biodegradable polymer consists of or comprises poly(lactide-co-glycolide) (PLGA).

8. The biodegradable polymeric microspheres of claim 7, where a ratio of lactide to glycolide units in the PLGA is between 60:40 and 40:60.

9. The biodegradable polymeric microspheres of claim 7, where a ratio of lactide to glycolide units in the PLGA is 50:50.

10. The biodegradable polymeric microspheres of claim 7, wherein the PLGA comprises acid terminated PLGA.

11. The biodegradable polymeric microspheres of claim 7, wherein the PLGA comprises a combination of acid terminated PLGA and ester terminated PLGA.

12. The biodegradable polymeric microspheres of claim 1, further comprising a pharmaceutical excipient selected from the group consisting of a preservative, a tonicity adjusting agent, a viscosity adjusting agent, a pH adjusting agent, a contrast agent, a detergent, or a sugar or sugar alcohol.

13. The biodegradable polymeric microspheres of claim 1, wherein the biodegradable polymeric microspheres are provided in dry form.

14. The biodegradable polymeric microspheres of claim 1, wherein the biodegradable polymeric microspheres are provided in an aqueous liquid.

15. The biodegradable polymeric microspheres of claim 1, wherein the biodegradable polymeric microspheres are provided in a vial.

16. The biodegradable polymeric microspheres of claim 1, wherein the biodegradable polymeric microspheres are provided in a syringe.

17. A method for the treatment of a patient having a solid tumor, comprising delivering to the solid tumor biodegradable polymeric microspheres that comprise a biodegradable polymer and an immunotherapeutic agent selected from an inhibitor of poly ADP ribose polymerase enzyme (PARP inhibitor) and/or a Toll-like receptor (TLR) agonist wherein (a) 50% of a total amount of the immunotherapeutic agent in the biodegradable polymeric microspheres is released from the biodegradable polymeric microspheres into a 37° C. solution of PBS Tween 20 (0.05%) at a point in time between 3 days and 14 days; (b) between 1 mg and 100 mg of immunotherapeutic agent per 1 g dry weight of microspheres is released from the biodegradable polymeric microspheres into a 37° C. solution of PBS Tween 20 (0.05%) at a point in time between 3 days and 14 days; or (c) both (a) and (b).

18. The method of claim 17, wherein the biodegradable polymeric microspheres are delivered by direct injection into the tumor.

19. The method of claim 17, wherein the biodegradable polymeric microspheres are delivered via one or more blood vessels feeding at least part of the tumor and wherein at least a portion of the biodegradable polymeric microspheres lodge in the blood vessels to provide an embolus.

20. A kit comprising
biodegradable polymeric microspheres that comprise a biodegradable polymer and an immunotherapeutic agent selected from an inhibitor of poly ADP ribose polymerase enzyme (PARP inhibitor) and/or a Toll-like receptor (TLR) agonist wherein (a) 50% of a total amount of the immunotherapeutic agent in the biodegradable polymeric microspheres is released from the biodegradable polymeric microspheres into a 37° C. solution of PBS Tween 20 (0.05%) at a point in time between 3 days and 14 days; (b) between 1 mg and 100 mg of immunotherapeutic agent per 1 g dry weight of microspheres is released from the biodegradable polymeric microspheres into a 37° C. solution of PBS Tween 20 (0.05%) at a point in time between 3 days and 14 days; or (c) both (a) and (b).
any one, any two, any three, any four, any five or all six of the following items: a syringe barrel, a vial, a needle, catheter, a guide wire, or an injectable liquid.
